(19) United States
(12) Patent Application Publication
(10) Pub. No.: US 2019/0290590 A1
(43) Pub. Date: Sep. 26, 2019

(54) INSTANT RELEASE CAPSULE BASED ON HOT MELT EXTRUDED POLYVINYL ALCOHOL

(71) Applicant: MERCK PATENT GMBH, Darmstadt (DE)

(72) Inventors: Mengyao ZHENG, Darmstadt (DE); Anja-Nadine KNUETTEL, Mannheim (DE)

(73) Assignee: MERCK PATENT GMBH, Darmstadt (DE)

(21) Appl. No.: 16/347,933

(22) PCT Filed: Nov. 1, 2017
(86) PCT No.: PCT/EP2017/077954

§ 371 Date: May 7, 2019

(30) Foreign Application Priority Data
Nov. 7, 2016 (EP) 16197614.7

(51) Int. Cl. A61K 9/48; A61K 9/16; A61K 47/32

(52) U.S. Cl. CPC A61K 9/4866; A61K 47/32; A61K 9/1635

(57) ABSTRACT

The present invention relates to an improved powdered extrudate based on polyvinyl alcohol (PVA), which can be used for pharmaceutical products, and that, due to its improved properties, can be better filled into capsules and the capsules show stable immediate drug release kinetic without problem of particle aggregation. Furthermore, this invention refers to pharmaceutical capsule composition comprising extruded polyvinyl alcohol as carrier matrix and can improve the solubility of API within immediate release kinetic. Moreover, the capsule show also benefit than tablet regarding to the material cost, because just PVA and API, no additional excipients are needed for capsule.

The present invention relates to a downstream formulation process of hot melt extrusion starting from an extrudate and including the end product, which are filled capsules with an improved milled extrudate powder based on polyvinyl alcohol (PVA), whereby said powder can be better filled into the capsule due to its improved properties. The produced capsules show a stable instant (immediate) release kinetic of the containing active ingredient without any problem of any particle aggregation.

### TECHNICAL FIELD

Solid dispersions are defined as being a dispersion of one or more active ingredients in an inert solid matrix and can broadly classified as those containing a drug substance in the crystalline state or in the amorphous state [Chiou W. L., Riegelman S. Pharmaceutical applications of Solid dispersion systems; J. Pharm Sci. 1971, 60 (9), 1281-1301]. In order to achieve a more consistent dosage rate of the active ingredient in pharmaceutical formulations, it is useful when the active ingredient is present as a homogeneous solid dispersion or solution in a carrier. Solid dispersions containing pharmaceutical active ingredients in the crystalline state provide dissolution enhancement by simply decreasing surface tension, reducing agglomeration, and improving wettability of the active substance [Sinswat P., et al.; Stabilizer choice for rapid dissolving high potency itraconazole particles formed by evaporative precipitation into aqueous solution; Int. J. of Pharmaceutics, (2005) 302; 113-124]. While crystalline systems are more thermodynamically stable than their amorphous counterparts, the crystalline structure must be destroyed during the dissolution process, requiring energy. Solid dispersions containing an active ingredient, this means a drug, dissolved at the molecular level, known as amorphous solid solutions, can result in a significant increase in dissolution rate and extent of supersaturation [DiNunzio J. C. et al. III Amorphous compositions using concentration enhancing polymers for improved bioavailability of itraconazole; Molecular Pharmaceutics (2008); 5(6):968-980]. While these systems have several advantages, physical instability can be problematic due to molecular mobility and the tendency of the drug to recrystallize. Polymeric carriers with high glass transition temperatures seem to be well suited to stabilize these systems by limiting molecular mobility.

As such, solid dispersions can be created by a number of methods, including, but not limited to, spray-drying, melt extrusion, and thermokinetic compounding.

Although hot melt extrusion (HME), a fusion processing technique, has been used in the food and plastics industry for more than a century, it has only recently gained acceptance in the pharmaceutical industry for the preparation of formulations comprising active ingredients processed by extrusion. And now, HME has been introduced as pharmaceutical manufacturing technology and has become a well-known process with benefits like continuous and effective processing, limited number of process steps, solvent free process etc.

During hot melt extrusion the active ingredients are mixed with and embedded in excipients, such as polymers and plasticizers. Furthermore, drug substances are exposed to elevated temperatures for a period of time. Although a variety of factors can affect the residence time distribution of an extruded substance, these times typically fall within the 1- to 2-min range (Breitenbach J., Melt extrusion: from process to drug delivery technology. Eur. J. Pharm. Biopharm. (2002), 54, 107-117).

Therefore, as carriers for the application of (hot) melt extrusion, the polymers should have suitable properties such us: thermoplasticity, suitable glass transition temperature or melting point, thermostability at required processing temperature, no unexpected chemical interaction with active ingredients etc. In this context, polyvinyl alcohol (PVA) is an excellent compound, which is suitable for (hot) melt extrusion, as carrier for pharmaceutically active ingredients. Polyvinyl alcohol (PVA) is a synthetic water-soluble polymer that possesses excellent film-forming, adhesive, and emulsifying properties. It is prepared from polyvinyl acetate, where the functional acetate groups are either partially or completely hydrolyzed to alcohol functional groups. As the degree of hydrolysis increases, the solubility of the polymer in aqueous media increases, but also the crystallinity of the polymer increases. In addition to this, the glass transition temperature varies depending on its degree of hydrolysis.

During hot melt extrusion, mixtures of active ingredients, thermoplastic excipients, and other functional processing aids, are heated and softened or melted inside of an extruder and extruded through nozzles into different forms. The obtained extrudate can be cut down into small beads or milled into fine powder. The milled extrudate powder can be compressed with other additional excipients for tableting, such us binders or disintegrants, to make the direct compression of tablets possible.

In this method, a thermoplastic polymer like PVA may be mixed with a pharmaceutical active ingredient (API). The mixture is fed into rotating screws that convey the powder into a heated zone where shear forces are imparted into the mixture, compounding the materials until a molten mass is achieved. The extrudate with solid dispersed API can be milled or pelletized into particles and filled into capsules. Hereby the solubility of a contained API can be improved in the final dosage form of the capsule.

U.S. Pat. No. 5,456,923 A provides a process for producing a solid dispersion, which overcomes disadvantages of the conventional production technology for solid dispersions. The process comprises employing a twin-screw extruder in the production of a solid dispersion. In accordance with this, a solid dispersion can be expediently produced without heating a drug and a polymer up to or beyond their melting points and without using an organic solvent for dissolving both components, and the resulting solid dispersions have excellent performance characteristics. The process claims a polymer that is natural or synthetic and can be employed as a raw material where the polymer's functions are not adversely affected by passage through the twin screw extruder.

EP 2 105 130 A1 describes a pharmaceutical formulation comprising a solid dispersion having an active substance embedded in a polymer in amorphous form, and an external polymer as a recrystallization inhibitor independently of the solid dispersion. The external polymer is claimed as a solution stabilizer. The active substance should be sparingly soluble or less sparingly soluble in water. Thermoplastic polymers are claimed as drug carriers to form a solid dispersion. It is claimed that the solid dispersion is obtained by melt extrusion. The process comprises melting and mixing the polymer and the active ingredient, cooling, grinding, mixing with the external polymer, and producing a pharmaceutical formulation. It is claimed that the melting is carried out at a temperature below the melting point of the drug. It is also claimed that the melting is carried out at a temperature above the T_{g} or melting point of the polymer, but from 0.1-5° C. below the melting point of the API. The melting point of pharmaceutical grades of PVA is normally above 178° C., although the glass transition temperature is in the range of 40-45° C.

### Problem to be Solved

It is known that extruded polyvinyl alcohol, which is approved for use in pharmaceutical formulations, is very difficult to mill into a readily flowable powder with fine uniformly shaped particles. However, if the particles of an active substance-containing powder are not fine enough, the active pharmaceutical ingredient dose (API löading) is limited, with which the volume of a gelatin capsule can be loaded even when larger capsules are used. Therefore it is an object of the present invention to provide a suitable, fine particulate, free-flowing polyvinyl alcohol powder.

But on the other hand, if the milled PVA particles are too fine, aggregation of these fine particles will happen, and in presence of moisture a gel layer on the surface of PVA aggregates is built and blocks the release of the containing API, and may promote re-crystallization of API, because the API in the super saturated dispersion of the aggregate tends thereto.

In addition, in tablet formulations with PVA as carrier for the active ingredients other binders and functional additives are required, usually in an amount of about 50% or more, based on the total weight of the completely compressed tablet. The high percentage of binder materials and other functional excipients limits the percentage of solid dispersion based on PVA, so that the drug loading efficiency is also limited.

The disintegration of PVA based tablets is normally very slow and lasts for several hours, in special cases sometimes more than 48 h. Therefore, a method for the preparation of PVA-based formulations is object of the present invention as well as to provide a specific final dosage form with instant release kinetic of active substance from the pharmaceutical formulation based on a PVA extrudate.

### SUMMARY OF THE INVENTION

Surprisingly it is found by experiments, that the capsule as final dosage form for hot melt extrusion compositions has the best performance for capsule filling and instant release of API, only if an extruded polyvinyl alcohol (PVA) powder is used, which is milled or pelletized into powder particles with particle sizes in the range of 500 μm to 3000 μm, preferably in the range of 500 μm to 1500 μm, most preferred in the range of 500 μm to 1000 μm, which shows improved flowability and excellent immediate drug release kinetic. Preferably, the PVA employed has to be melt extruded or hot-melt extruded prior to milling or pelletizing. PVA grades having a viscosity ≤40 mPa·s, the viscosity being measured on 4% w/v aqueous solution at 20° C. DIN, are particularly suitable for the production of these PVA powders. Polyvinyl alcohol grades fulfilling these conditions are preferably selected from the group: PVA 2-98, PVA 3-80, PVA 3-83, PVA 3-85, PVA 3-88, PVA 3-98, PVA 4-85, PVA 4-88, PVA 4-98, PVA 5-74, PVA 5-82, PVA 5-88, PVA 6-88, PVA 6-98, PVA 8-88, PVA 10-98, PVA 13-88, PVA 15-79, PVA 15-99, PVA 18-88, PVA 20-98, PVA 23-88, PVA 26-80, PVA 26-88, PVA 28-99, PVA 30-75, PVA 30-92, PVA 30-98, PVA 32-80, PVA 32-88, PVA 40-88, most preferred from the group: PVA 3-88, PVA 4-88, PVA 5-74, PVA 5-88, PVA 8-88, and PVA 18-88.

Subject matter of the present invention is therefore a powdery composition as characterized here for the preparation of immediate release capsule formulations, comprising extruded polyvinyl alcohol as carrier, which is extruded and homogeneously milled with at least one active pharmaceutical ingredient (API), whereby this milled powder is storage and transport-stable, showing an improved flowability, and leading to an immediate drug release process without any problem of particle aggregation and re-crystallization.

While for the particle sizes ≤500 μm without any other additional excipients, undesired aggregation of particles and API re-crystallization will happen during the dissolution process, surprisingly it was found, that if an inorganic salt powder is mixed with the PVA powder during extrusion and if this mixture is filled into a capsule, the aggregation of PVA particle will be blocked and the capsule can deliver a stable instant release of the contained API. Here the concentration of the added inorganic salt depends on the type of the comprising API, and in general it is added in an amount of 0.5 to 20% by weight to a powdery composition having particle sizes in the range of ≤500 μm.

Accordingly, a PVA grade is subject matter of the present invention, which is suitable as thermoplastic polymer for HME and which is also suitable for one of the downstream formulation process of HME leading to a pharmaceutical powder composition which is filled into capsules. In this process polyvinyl alcohol is extrusion-treated and homogeneously milled or pelletized together with at least one pharmaceutical ingredient (API) to a powder, which is dosed into capsules.

In a further embodiment of the invention polyvinyl alcohol as described above is extruded with at least one active pharmaceutical ingredient and milled homogeneously, whereby the resulting milled particles are storage and transport-stable, and show a suitable flowability for capsule filling. The resulting capsule formulation shows a stable instant drug release kinetic without any aggregation problem during the dissolution.

Compared with compressed tablets, the benefit of capsules is the simpler manufacturing process and less material costs, because no additional additives are needed to be added together with the milled extrudate, if the extrudate particle is milled to a particle size of 500 μm to 3000 μm, preferably in the range of 500 μm to 1500 μm, most preferred in the range of 500 μm to 1000 μm.

Said method or process for producing the pharmaceutical capsules of the present invention is characterized in that the extrudate of ingredients including polyvinyl alcohol and at least one API as characterized above is processed by homogeneously milling together into beads or particles with defined particle size, and which is then filled directly into capsules. If needed and if PVA powders are applied having particle sizes ≤500 μm, PVA is milled together with at least one API and at least one inorganic salt resulting in a stable powder, which is dosed into capsules.

The particular advantage of the present invention is that the obtained milled extrudate particle can be directly filled into capsules. The best particle size is also defined to deliver a stable instant release kinetic of the comprising drug without any problem of aggregation. According to the present invention the process for producing the final dosage form includes the steps of
a) (cryo-) milling or pelletizing the extrudate of polyvinyl alcohol (PVA) and at least one API to particles having particle sizes in the range of 500 μm-3000 μm, preferably in the range of 500 μm to 1500 μm, most preferred in the range of 500 μm to 1000 μm, or
b) for extruded and milled polyvinyl alcohol (PVA) powder having particle size ≤500 μm, additionally at least one inorganic salt powder in an amount of 0.5 to 20% by weight, depending on the nature of the comprising API type, is needed to be mixed with the extrudate powder to avoid the aggregation of PVA particle and of API re-crystallisation effectively,
   and
c) feeding this powdery composition evenly into capsules.
If needed, further additives may be added during extruding, milling or pelletizing.

This process can be performed particularly well, if in a) polyvinyl alcohol (PVA) based extrudate is milled to a powder having a particle size in the range of 500 μm-3000 μm, preferably in the range of 500 μm to 1500 μm, most preferred in the range of 500 μm to 1000 μm, namely when solid polyvinyl alcohol (PVA) having pharmaceutical grade is applied which is characterized having a viscosity ≤40 mPa·s, the viscosity being measured on 4% aqueous solution at 20° C. DIN 53015. In this case very particularly preferred is the use of polyvinyl alcohol (PVA), selected from the group: PVA 2-98, PVA 3-80, PVA 3-83, PVA 3-85, PVA 3-88, PVA 3-98, PVA 4-85, PVA 4-88, PVA 4-98, PVA 5-74, PVA 5-82, PVA 5-88, PVA 6-88, PVA 6-98, PVA 8-88, PVA 10-98, PVA 13-88, PVA 15-79, PVA 15-99, PVA 18-88, PVA 20-98, PVA 23-88, PVA 26-80, PVA 26-88, PVA 28-99, PVA 30-75, PVA 30-92, PVA 30-98, PVA 32-80, PVA 32-88, PVA 40-88, most preferred from the group: PVA 3-88, PVA 4-88, PVA 5-74, PVA 5-88, PVA 8-88, and PVA 18-88.

Thus, a capsule composition from PVA extrudate, which is characterized as disclosed herein and which is obtainable by a process as characterized here, is the subject of the present invention.

### DETAILED DESCRIPTION OF THE INVENTION

While the making and using of various embodiments of the present invention are discussed in detail below, it should be appreciated that the present invention provides more applicable inventive concepts than described here in detail. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “a homogenous melt, or mixture or form” refers to the various compositions that can be made by extruding the made-up source material, which is prepared by milling or pelletizing.

As used herein, the term “heterogeneously homogeneous composite” refers to a material composition having at least two different materials that are evenly and uniformly distributed throughout the volume and which are prepared of the one or more APIs and the one or more pharmaceutically acceptable excipients, including a pretreated PVA into a composite.

As used herein, “bioavailability” is a term meaning the degree to which a drug becomes available to the target tissue after being administered to the body. Another meaning of this term and which is also meant here is the rate and extent to which the active ingredient or active moiety is absorbed from a drug product and becomes available at the site of action. Poor bioavailability is a significant problem encountered in the development of pharmaceutical compositions, particularly those containing an active ingredient that is not highly soluble.

As used herein, the phrase “pharmaceutically acceptable” refers to molecular entities, compositions, materials, excipients, carriers, and the like that do not produce an allergic or similar untoward reaction when administered to humans in general.

As used herein, “pharmaceutically acceptable carrier” or “pharmaceutically acceptable materials” includes any and all solvents, dispersion media, coatings, antibacterial and antifungal agents, isotonic and absorption delaying agents, and the like. The use of such media and agents for pharmaceutical active substances is well known in the art.

The API (active pharmaceutical ingredient) may be found in the form of one or more pharmaceutically acceptable salts, esters, derivatives, analogs, prodrugs, and solvates thereof. As used herein, a “pharmaceutically acceptable salt” is understood to mean a compound formed by the interaction of an acid and a base, the hydrogen atoms of the acid being replaced by the positive ion of the base.

As used herein, “poorly soluble” refers to having a solubility means the substance needs ≥100 ml solvent to dissolve 1 g substance.

A variety of administration routes are available for delivering the APIs to a patient in need. The particular route selected will depend upon the particular drug selected, the weight and age of the patient, and the dosage required for therapeutic effect. The pharmaceutical compositions may conveniently be presented in unit dosage form. The APIs suitable for use in accordance with the present disclosure, and their pharmaceutically acceptable salts, derivatives, analogs, prodrugs, and solvates thereof, can be administered alone, but will generally be administered in admixture with a suitable pharmaceutical excipient, diluent, or carrier selected with regard to the intended route of administration and standard pharmaceutical practice.

The excipients and adjuvants that may be used in the presently disclosed compositions and composites, while potentially having some activity on their own, for example, antioxidants, are generally defined for this application as compounds that enhance the efficiency and/or efficacy of the effective ingredients. It is also possible to have more than one effective ingredient in a given solution, so that the particles formed contain more than one effective ingredient.

As stated, excipients and adjuvants may be used to enhance the efficacy and efficiency of the APIs dissolution.

Depending on the desired administration form the formulations can be designed to be suitable in different release models, which are well known to the skilled person, as there are: immediate, rapid or extended release, delayed release or for controlled release, slow release dosage form or mixed release, including two or more release profiles for one or more active pharmaceutical ingredients, timed release dosage form, targeted release dosage form, pulsatile release dosage form, or other release forms.

The resulting composites or compositions disclosed herein may also be formulated to exhibit enhanced dissolution rate of a formulated poorly water soluble drug.

The United States Pharmacopeia-National Formulary mandates that an acceptable polyvinyl alcohol for use in pharmaceutical dosage forms must have a percentage of hydrolysis between 85 and 89%, as well as a degree of polymerization between 500 and 5000. The degree of polymerization (DM) is calculated by the equation:

DM=(Molar Mass)/((86)−(0.42(the degree of hydrolysis)))

The European Pharmacopoeia mandates that an acceptable polyvinyl alcohol for use in pharmaceutical dosage forms must have an ester value no greater than 280 and a mean relative molecular mass between 20,000 and 150,000. The percentage of hydrolysis (H) can be calculated from the following equation:

H=((100−(0.1535)(EV))/(100−(0.0749)(EV)))×100

Where EV is the ester value of the polymer. Thus, only polymers with a percentage of hydrolysis greater than 72.2% are acceptable according to the European Pharmacopoeia monograph.

As already mentioned above, commercial polyvinyl alcohols in particulate form have poor flow behavior, especially if they are characterized by low viscosities (measured in a 4% aqueous solution at 20° C.). Accordingly, these powders have no continuous trouble-free flow. However, the latter is a prerequisite for a uniform feed to the processing of such powder materials.

Theoretically, powders, whose particle shapes are rather round and spherical, in general have the best flow behavior. Accordingly, in the past, attempts have been made to produce polyvinyl alcohol powders already directly by its synthesis with spherical particles. For example, from DE 38 11 201A a method is known for producing of spherical particles by suspension polymerization. However, this reaction requires a special adjustment of the reaction conditions. In addition, this reaction has to be followed by a hydrolysis reaction. With different particle sizes, it is difficult to achieve a uniform degree of hydrolysis of the polymer particles. By this method, polyvinyl alcohol powders are produced having viscosities of 80 mPa·s or higher.

Therefore, for the production of polyvinyl alcohol powders, which are comparable with those of the present invention, this method provides no alternative, especially as here PVA grades are desirable having viscosities of ≤40 mPa·s.

Now, it has been found that these polyvinyl alcohol grades having viscosities of ≤40 mPa·s are also suitable to be manufactured by melt extrusion if they are pretreated as disclosed in the following and a homogenously dispersed solid solution of pharmaceutical active ingredient in polyvinyl alcohol can be produced by extrusion.

In this way also poorly soluble pharmaceutical active ingredients (from BCS class II and IV) can be homogeneously mixed with PVA to build a solid dispersion. Furthermore, it was found by experiments that PVA in the different degrees of hydrolysis having viscosities of ≤40 mPa·s can be homogeneously mixed by melt extrusion with poorly soluble active ingredients, especially with PVA that is in accordance with the European Pharmacopoeia monograph and which is a pharmaceutically acceptable PVA with hydrolysis grades greater than 72.2%, and especially which includes grades of PVA that are pharmaceutically acceptable by either the USP (hydrolysis between 85-89%) or Ph. Eur. (hydrolysis grades greater than 72.2%). These PVA qualities have a molecular weight in the range of 14,000 g/mol to 250,000 g/mol.

Milled or pelletized compositions according to the invention may comprise at least a biologically active ingredient combined with a PVA that is pharmaceutically acceptable, which is combined with another pharmaceutically acceptable polymer. Such pharmaceutically acceptable polymer can also be selected from the group of hydrophilic polymers and can be a primary or secondary polymeric carrier that can be included in the composition disclosed herein and including polyethylene-polypropylene glycol (e.g. POLOXAMER™), carbomer, polycarbophil, or chitosan, provided that they are as free-flowing powder and are extrudable polymers. Hydrophilic polymers for use with the present invention may also include one or more of hydroxypropyl methylcellulose, carboxymethylcellulose, hydroxypropyl cellulose, hydroxyethyl cellulose, methylcellulose, natural gums such as gum guar, gum acacia, gum tragacanth, or gum xanthan, and povidone. Hydrophilic polymers also include polyethylene oxide, sodium carboxymethycellulose, hydroxyethyl methyl cellulose, hydroxymethyl cellulose, carboxypolymethylene, polyethylene glycol, alginic acid, gelatin, polyvinylpyrrolidones, polyacrylamides, polymethacrylamides, polyphosphazines, polyoxazolidines, poly(hydroxyalkylcarboxylic acids), carrageenate alginates, carbomer, ammonium alginate, sodium alginate, or mixtures thereof.

In general, it must be considered that there are special requirements for polymers used as hot melt extrusion excipients:

The polymer must be thermoplastic, must have a suitable glass transition temperature and a high thermal stability. The polymer must have no toxic properties and must have a high biocompatibility, etc. Therefore, pharmaceutical grades of polyvinyl alcohol (PVA), which are chosen here for the preparation of formulations comprising active ingredients by hot melt extrusion, are those having a low viscosity.

Moreover, for one of the downstream formulation of hot melt extrusion, the capsule, not all of the particle ranges are suitable to be filled into capsules: on one hand, if the particle is not fine enough, the API dose (API loading) within the capsule will be limited because of the volume of the particles loaded with API.

On the other hand, if the milled PVA particle is too fine, aggregation of these fine particles and aggregation-induced API re-crystallization will happen. The gel layer on the surface of PVA aggregates blocks the release of API, and may promote re-crystallization of API, because the API suffers under a super saturated state inside of the aggregate. Therefore, the extrudate should be milled into particles with suitable particle size and distribution.

Polyvinyl alcohol (PVA) is a synthetic polymer, which is produced by polymerization of vinyl acetate and partial hydrolysis of the resulting esterified polymer. As already mentioned above, chemical and physical properties of polyvinyl alcohol, such as viscosity, solubility, thermal properties, etc. are very depending on its degree of polymerization, chain length of PVA polymer, and the degree of hydrolysis.

PVA can be used for the production of different formulations for various modes of administration to treat a variety of disorders. Accordingly, PVA is processed in a wide range of pharmaceutical dosage forms, including ophthalmic, transdermal, topical, and especially, oral application forms.

As mentioned above, it also is necessary for the successful industrial processing of a solid dosage form in

1.) an extrusion process,

2.) a milling or pelletizing process, and

3.) for filling into capsules,

that a uniform continuous metering is possible into the extruder, miller or pelletizer and into the capsule filling machine.

By experiments it is found here, that for producing capsules as downstream formulation of extrusion based on PVA the milled extrudate must have suitable particle characteristics, including appropriate particle sizes, flowability or fluidity. It is also found, that milled extrudate based on polyvinyl alcohol of pharmaceutical grade to a powder as characterized above and having particle sizes in the range of 500 μm-3000 μm, preferably in the range of 500 μm to 1500 μm, most preferred in the range of 500 μm to 1000 μm, has the best performance for capsule filling and instant release of API. In this case very particularly preferred is the use of polyvinyl alcohol (PVA) having pharmaceutical grade, selected from the group: PVA 2-98, PVA 3-80, PVA 3-83, PVA 3-85, PVA 3-88, PVA 3-98, PVA 4-85, PVA 4-88, PVA 4-98, PVA 5-74, PVA 5-82, PVA 5-88, PVA 6-88, PVA 6-98, PVA 8-88, PVA 10-98, PVA 13-88, PVA 15-79, PVA 15-99, PVA 18-88, PVA 20-98, PVA 23-88, PVA 26-80, PVA 26-88, PVA 28-99, PVA 30-75, PVA 30-92, PVA 30-98, PVA 32-80, PVA 32-88, PVA 40-88, most preferred from the group: PVA 3-88, PVA 4-88, PVA 5-74, PVA 5-88, PVA 8-88, and PVA 18-88, which is extruded with at least one API and further milled to a powder with particles in the range of 500 μm-3000 μm, preferably in the range of 500 μm to 1500 μm, most preferred in the range of 500 μm to 1000 μm. The milled extrudate powders, comprising particles larger than in the range of about 3000 μm (d₅₀), are not suitable for capsule filling because of their limitation of drug dose.

Now, for producing the free-flowing, readily dosable powder, PVA is physically blended with the active ingredients in an amount of 20-60% by weight, with or without additional plasticizers and optionally with further additives. Then the mixture is extruded under suitable conditions depending on the added APIs. After extrusion the received product is milled or pelletized into powders with different particle sizes, which in turn affect the flowability, homogeneity and dissolution properties.

It was also found through the experiments, that for particle sizes ≤500 μm without any other additional excipients, aggregation of particles and re-crystallization will happen during the dissolution process. However, the experiments surprisingly have shown, that if inorganic salt (concentration depends on the API type, normally 0.5-20% by weight) is mixed together with the extrudate powder and filled together into the capsule, the aggregation of PVA particles will be blocked and the capsule can deliver a stable instant release of the comprising API. In this case 0.5-20% by weight of inorganic salt is needed to be added to improve the dissolution and to avoid particle aggregation. But depending on the properties of the comprising active ingredient, the corresponding effect can also be achieved by adding a smaller amount of salt. In some cases, however, more salt may also be required. Suitable salts for this purpose are physiologically acceptable salts such as, sodium carbonate, potassium bicarbonate, sodium chloride, magnesium carbonate. However, it is also possible to use other physiologically acceptable inorganic salts which produce the same effect.

In summary, surprisingly, it is found that compositions based on PVA are suitable to be filled into capsules, if:
1. this capsule composition is based on milled or pelletized PVA/API extrudate having particle sizes in the range of 500 μm-3000 μm, preferably in the range of 500 μm to 1500 μm, most preferred in the range of 500 μm to 1000 μm.
2. the particle size is 500 μm, and if the composition comprises 0.5-20% by weight or more of at least one inorganic salt and if this composition is added into the extrudate powder.

### Examples

Even without any further explanations, it is assumed that a person skilled in the art can make use of the above description in its widest scope. The preferred embodiments and examples are therefore to be regarded merely as descriptive but in no way limiting disclosures.

For better understanding and for illustration, examples are given below which are within the scope of protection of the present invention. These examples also serve for the illustration of possible variants.

The complete disclosure of all applications, patents and publications mentioned above and below are incorporated by reference in the present application and shall serve in cases of doubt for clarification.

It goes without saying that, both in the examples given and also in the remainder of the description, the quoted percentage data of the components present in the compositions always add up to a total of 100% and not more. Given temperatures are measured in ° C.

Now, in order to carry out the following experiments, extrudate with PVA and API was milled/pelletized into four charges under different milling conditions (definition of method is following) to obtain different particle sizes and particle distributions of extrudate powders:

Charge 1: Particle size in the range of 500 μm

Charge 2: Particle size in the range of about 500 μm

Charge 3: Particle size in the range of about 1500 μm

Charge 4: Particle size in the range of about 3000 μm

Before milling, PVA was physically blended with active ingredients in an amount of 20-60% by weight, with or without additional plasticizers. The mixture was extruded under suitable conditions (depends on API) and milled or pelletized into different particle size, which is characterized regarding to the flowability, homogeneity and dissolution.

The analysis of the data obtained indicated, that milled PVA particles in the range of 500 μm-3000 μm, preferably in the range of 500 μm to 1500 μm, most preferred in the range of 500 μm to 1000 μm, has the best performance for capsule filling and instant release of API. If the particle 500 μm, 0.5-20% by weight (not limited) inorganic salt is needed to be added into the extrudate powder to make the instant release of drug possible without any problem like aggregation and API re-crystallization.

Methods and Materials

1. Raw Materials and Manufacturing Method

1.1 Materials

Raw Material:
Poly vinyl alcohol 4-88, excipient EMPROVE® exp Ph Eur, USP, JPE, Article No. 1.41350, Merck KGaA, Darmstadt, Germany
Itraconazole, active ingredient, Selectchemie, AG, Germany
KHCO₃, Merck KGaA, Darmstadt, Germany
NaCl, Merck KGaA, Darmstadt, Germany

1.2 Experiments and Characterization Methods

1.2.1 Extrusion Process

Equipment and Extrusion Process:
Physical blend of composition for hot melt extrusion, including active ingredients: TURBULA® Shaker-Mixer
Brabender® Mini-Compounder KETSE 12/36 D
The mixture of PVA and active ingredient were blended using TURBULA® Shaker-Mixer homogeneously (the concentration of polymer and active ingredient depends on the types and physical properties of them). The mixture was then loaded into the extruder with well designed extrusion parameters, such as feeding rate, screw design, screw speed, extrusion temperature etc. The set up of those parameters depend also on the types and physical properties of polymer and active ingredients.

1.2.2 Milling or Pelletizing Process
Equipment in lab: Ultra-Zentrifugalmühle ZM 200 200-240V, 50/60 Hz
Scale up equipment for milling: Mill equipment for extrudate milling: aeroplex spiral jet mill, type 200 AS Hosokawa Alpine, Augsburg, Germany
Brabender® Pelletizer

Milling Conditions:

with liquid nitrogen as cold grinding. The desired particle size is produced empirically in particular by varying the grinding temperature, to control the particle size of PVA. The grinding conditions are varied until the desired particle size is obtained.

Pelletizing Condition: The Pelletizer can be Set Up to Produce Desired Particle from 500 μm to 7000 μm.

Obtained Particle Groups:
Charge 1: Particle size in the range of 500 μm (d₅₀) (produced by cryo-milling)
Charge 2: Particle size in the range of about 500 μm (produced by pelletizing)
Charge 3: Particle size in the range of about 1500 μm (produced by pelletizing)
Charge 4: Particle size in the range of about 3000 μrn (produced by pelletizing)

Particle Size and Distribution Analysis

Particle size determination by laser diffraction with dry dispersion: Mastersizer 2000 with dispersing Scirocco 2000 (Malvern Instruments Ltd. UK.), Provisions at 1, 2 and 3 bar backpressure; Evaluation Fraunhofer; Dispersant RI: 1000, obscuration limits: 0.1-10.0%, Tray Type: General Purpose, Background Time: 7500 msec Measurement Time: 7500 msec, implementation in accordance with ISO 13320-1 and the details of the technical manual and the specifications of the equipment manufacturer; Information in Vol-%.

1.2.3 Dissolution

For the real time dissolution performance, the following equipments are used:

System 1:
Sotax AT 7 on/offline
Pumpe CY-7-50
Fraktionssammler: C613 14 Kanal 3 Wege Ventilbalken für Reagenzgläser
Agilent 8453 Photometer

System 2
Sotax AT 7 on/offline
Pumpe CP 7-35
Fraktionssammler: C 613 14 Kanal 3 Wege Ventilbalken für Vials
Photometer Analytik Jena Specord 200 plus

2. Research Results

2.1 Particle Size and Distribution

A milled extrudate powder having this particle size distribution is characterized by the logarithmic plot of particle sizes ranging up to 100 microns to their volume percentage:

TABLE 1
particle size and distribution of milled extrudate
with 30% itraconazole and 70% PVA

| | Dv5 | Dv10 | Dv20 | Dv25 | Dv30 | Dv50 | Dv75 | Dv90 | Dv95 |
|---|---|---|---|---|---|---|---|---|---|
| | (μm) | (μm) | (μm) | (μm) | (μm) | (μm) | (μm) | (μm) | (μm) |
| Group 1 | 13.18 | 22.07 | 30.15 | 59.15 | 59.88 | 97.03 | 156.04 | 221.73 | 265.33 |

The groups 2, 3, 4 were produced by pelletizer and therefore have no Gauss-Distribution.

2.2 Relationship Between Particle Size and Dissolution Performance
1. Groups 2, 3, 4 (with particle size of 500 μm, 1500 μm and 3000 μm) show very similar dissolution performance of itraconazole: all of them are immediate release and achieve a 100% dissolution after 60 min.

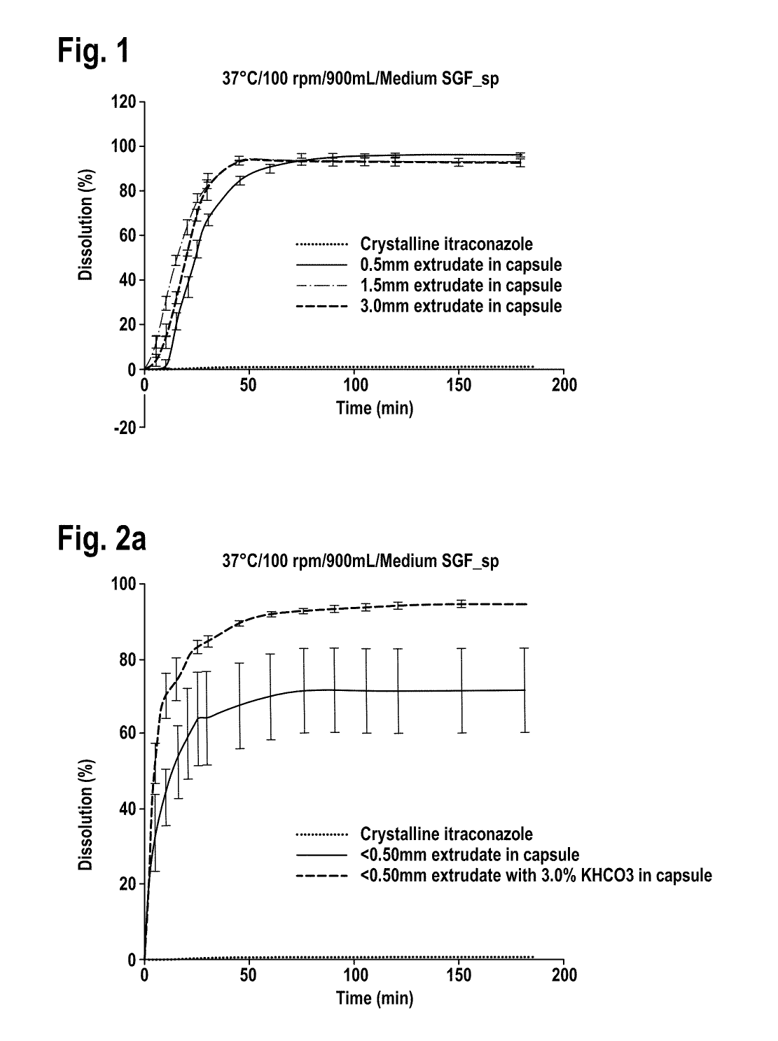

FIG. 1: instant release of itraconazole extrudate in capsule with different particle size
2. If fine micronized particles are required, the aggregation problem can be solved: with addition of salt to avoid the aggregation of fine particle, which is micronized and <0.30 mm.

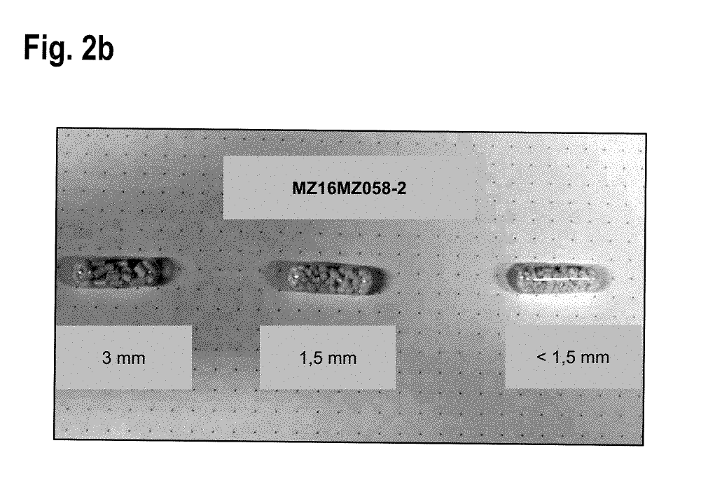

FIG. 2a: Dissolution performance of capsules, which are filled with micronized particles (<0.50 μm), with and without additional 3% inorganic salt.

FIG. 2b: The photo (1) shows capsules with filled differently sized particles based on PVA and itraconazole extrudate.

2.3 Summary

### Advantages of the Present Invention

1. The method to mill/pelletize the extruded PVA/API into best particle size for capsule filling.
2. The advantages of the best particle size and distribution of milled or pelletized PVA/API extrudate: excellent flowability and feasibility of capsule filling and the capsule shows excellent immediate drug release kinetic.
3. The method to avoid the aggregation of fine milled particle based on PVA during the dissolution process.
4. Down stream process with capsule to save the material cost

1. Extruded polyvinyl alcohol (PVA) powder, which is milled or pelletized into powder particles with particle sizes in the range of 500 μm to 3000 μm, preferably in the range of 500 μm to 1500 μm, most preferred in the range of 500 μm to 1000 μm, with improved flowability and excellent immediate drug release kinetic.

2. Polyvinyl alcohol according to claim 1, which is melt extruded or hot melt extruded before milling or pelletizing.

3. Polyvinyl alcohol according to claim 1 having a viscosity ≤40 mPa·s, the viscosity being measured on 4% w/v aqueous solution at 20° C. DIN 53015.

4. Polyvinyl alcohol according to claim 1, which is selected from the group PVA 2-98, PVA 3-80, PVA 3-83, PVA 3-85, PVA 3-88, PVA 3-98, PVA 4-85, PVA 4-88, PVA 4-98, PVA 5-74, PVA 5-82, PVA 5-88, PVA 6-88, PVA 6-98, PVA 8-88, PVA 10-98, PVA 13-88, PVA 15-79, PVA 15-99, PVA 18-88, PVA 20-98, PVA 23-88, PVA 26-80, PVA 26-88, PVA 28-99, PVA 30-75, PVA 30-92, PVA 30-98, PVA 32-80, PVA 32-88, PVA 40-88, preferably selected from the group PVA 3-88, PVA 4-88, PVA 5-74, PVA 5-88, PVA 8-88, and PVA 18-88.

5. A powdery composition for the preparation of immediate release capsule formulations, comprising extruded polyvinyl alcohol according to claim 1 as carrier, which is extruded and homogeneously milled with at least one active pharmaceutical ingredient (API), whereby this milled powder is storage and transport-stable, showing an improved flowability, and leading to an immediate drug release process without any problem of particle aggregation and re-crystallization.

6. A powdery composition for the preparation of immediate release capsule formulations, comprising extruded polyvinyl alcohol according to claim 2 as carrier, which is extruded and homogeneously milled together with at least one active pharmaceutical ingredient (API) and additionally with inorganic salt in an amount of 0.5 to 20% by weight to a powdery composition having particle sizes in the range of ≤500 μm.

7. A process for the preparation of pharmaceutical preparations in form of capsules, characterized in that a powdery composition of ingredients including the powdered polyvinyl alcohol according to claim 1 is prepared and filled into capsules.

8. A process according to claim 7 for the preparation of pharmaceutical preparations in form of capsules, characterized in that the extrusion-treated and milled or pelletized powdery polyvinyl alcohol is homogeneously milled together with at least one pharmaceutical ingredient (API) and the resulting powder dosed into capsules.

9. A process according to claim 7 for the preparation of pharmaceutical preparations in form of capsules, characterized in that the powdery polyvinyl alcohol is homogeneously milled together with at least one pharmaceutical ingredient (API) and at least one inorganic salt resulting in a stable powder, which is dosed into capsules.

10. A process according to claim 7 for the preparation of pharmaceutical preparations in form of capsules with immediate drug release kinetic, characterized in that powdery polyvinyl alcohol is homogeneously milled together with at least one pharmaceutical ingredient (API) and at least one inorganic salt in an amount of 0.5 to 20% by weight and optionally further additives resulting in a powdery composition without any problem of particle aggregation and recrystallization, having particle sizes in the range of ≤500 μm, and having improved flowability, and which is then dosed into capsules.
